(19) United States
(12) Patent Application Publication
(10) Pub. No.: US 2013/0060183 A1
(43) Pub. Date: Mar. 7, 2013

(54) Attachable grip enhancers and uses and methods of said enhancers

(71) Applicant: John Cuevas Ramirez, Redlands, CA (US)

(72) Inventor: John Cuevas Ramirez, Redlands, CA (US)

(21) Appl. No.: 13/199,530

(22) Filed: Sep. 2, 2011

(51) Int. Cl. A61F 13/10; A63B 57/00; A63B 69/00; B32B 7/12; B65G 7/00; B32B 3/26; A61L 15/44; B32B 3/02; C09J 7/02; A63B 69/38; B32B 3/30

(52) U.S. Cl. 602/48; 473/422; 473/409; 602/54; 428/343; 428/172; 428/317.3; 428/66.2; 294/25; 428/80

(57) ABSTRACT

According to the various features, characteristics and embodiments of the present invention which will become apparent as the description thereof proceeds, the present invention provides attachable grip enhancers for general use, as well as in playing the game of various sports, that could be attached to an individual, and uses and methods thereof. The present invention and its embodiments can, among other things, enhance the overall performance in arm tasks conducted by the wrist, forearm and/or bicep areas, as well as hand tasks.

### FIELD OF THE INVENTION

The present invention relates to general use apparatus, and uses thereof, used in general use activities, as well as in playing the game of various sports. The present invention and its embodiments can, among other things, enhance the overall performance in arm tasks conducted by the wrist, forearm and/or bicep areas, as well as hand tasks. These embodiments are attached to an individual thereby providing, among other thing, said individual with enhanced grip and/or control capabilities. General use embodiments may also be secondarily attached to an object, device or ball for localized grip enhancing.

### BACKGROUND OF THE INVENTION

Grip and control enhancers have come a long way in assisting one to complete a task or goal. Many tasks, for example, may be divided into arm tasks and hand tasks, and successful completion of said tasks often require the ability to properly grip or control an object.

Arm task enhancers could certainly assist an individual more effectively perform non-sport activities.

One non-sport activity in need of arm task enhancers is commonly conducted by waiters. For example, a waiter (or other food service provider) often has to use a bus tray, placing it on his forearm while gripping the bus tray with his hand. This is especially the case when the waiter is standing next to a guest who is being somehow served. Although the hand provides a solid grip of the bus tray, the bus tray can become unstable at times, particularly when either placing items on the bus tray or removing them from the bus tray. Additionally, if this activity creates a significant weight change on the bus tray, the waiter could find great difficulty in maintaining control. The result, usually, is for the waiter to quickly use his other hand to retain control and balance. This problem, however, will remain until the food server is able to somehow increase his overall control of the bus tray by being better able to grip the bus tray with his arm.

Arm task enhancers could also assist an individual complete a sport object. An important goal in playing sports, for example, is to win. Often that means proper play execution, proper form in the sports fundamentals and especially good ball control. Many of these tasks are performed by the hand, but are often also performed, to some degree, with parts of the arm (the wrists, forearm, elbow and bicep areas). Ball control and therefore proper play execution depend on adequately using the parts of the forearm, elbow and/or bicep areas to handle, strike, control or otherwise maintain possession. Inadequate play execution can result in inconsistencies and turnovers, both long standing problems in many sports, and can often determine the outcome of a game.

In the sport of football, for example, lack of play execution is often categorized by turnovers, fumbles and incomplete passes. This is of particular concern to those players that have to control a football such as running backs, receivers, tight ends, kick returners, punt returners and even quarterbacks. Athletes that play any of these positions are often asked, in some way, to run, catch, throw or otherwise control a football. Successful play execution—which in the case of football includes minimizing fumble, incompletes and inconsistencies—can often be the difference in the successful outcome of a team's objective. Creating and maintaining a solid and stable control of the ball is therefore essential in proper play execution and performance.

In football, as in many other sports, controlling a ball is often done not just by using their hands, but by using other parts of the body as well.

A football running back, for instance, might be particularly concerned with not fumbling the ball. A running back's performance is measured not only by his yards per carry but also in his ability to minimize his fumbles. Unfortunately, one need only view the statistics to see that fumbles persist as an insoluble problem, even at the professional level today.

Part of the problem lie in the seemingly inherently unstable way a player controls and cradles a ball when running. Proper ball handling technique is to grab one end of the football with your hand, and then resting the ball on the forearm of the same arm. As you begin to run with the ball, you may also place the opposite end of the ball (the end that is not being held by the hand) in the inside elbow area, between the forearm and bicep, and the ball is almost always touching the wrist area. Although prior art exists to increase grip around the hand, no art currently exists that will increase ones grip in the general arm area, such as the wrist, forearm, elbow and bicep areas.

Football players who catch a football (hereinafter called ‘receivers’) might be particularly concerned with making a catch and completing a reception, and being able to control a ball with the arm area. Enough skill and precision must take place in order to get the ball from the quarterback to a receiver; timing, stable footing, and protecting the ball just to name a few. When a receiver first catches a ball, he usually brings the ball into the arm, thus holding the ball with the hand, as well as the forearm and elbow area. No art exists to enhance the elbow grip or the elbow area, an area that plays a significant role in proper play execution. Passing the ball is a significant part of the sport of football, sometimes throwing as much as 103 times in a game (Seattle vs. San Diego, 2002). Thus, developing a solution to enhance one's ability of better controlling a catch and completing a pass reception would substantially impact a sport.

Additionally, inconsistencies, or incompletes, often arise when a ‘stress factor’ is introduced once a receiver first touches and places the ball on their arm area. This problem is so pronounced that many instant replay situations revolve around seeing whether the ball moves even slightly in the receivers arm—including the forearm which has no real grip enhancers. As such, an unrecognized problem currently exists because it is very difficult to grip a ball with parts of the arm.

Clearly, maintaining good ball control is important. In football, unstable or weak ball control can, among other things increase fumbles, increase incompletes and thereby increase turnovers and decrease performance.

There have been some attempts through the years to solve the problems of inconsistencies and turnovers in the sport of football. For example, changes have been made to the actual football in order to make the ball easier to handle. Changes to the shape and size, as well as the addition of grip enhancing materials to the ball—such as the addition of PVC dots—have made it possible to make the ball more gripable. The ability of the player to maintain control of the football was still problematic because of the lack of any grip enhancing device for the player to use; devices that could be placed on the arm such that the player could now more significantly control a ball with his arm, thereby creating an overall grip of the football throughout the football. As a result of this unmet need, inconsistencies and turnovers were still high in the sport.

The introduction and subsequent proliferation in the use of gloves found some success but even with these advancements, however, fumbles and incompletes still persist today. For one, whereas a running back who uses a grip enhancing glove will be better able to properly grip a football by using his hands, his hand, and therefore the grip enhancing device, only cover the front part of the ball, leaving the rest of the ball, and therefore the overall grip, still significantly unstable. One need only add a stress factor and this currently unstable hold on the ball can easily result in a fumble.

Good ball control is so important in football that inventions were created and widely used to enhance the gripping abilities of an individual's hand. Unfortunately, the grip improvements stopped with the hand gloves. No improvements have been developed to help the rest of the arm better handle a ball. No improvements have been developed that could provide enhanced gripping support around the forearm area, an area that plays a major role in maintaining control of a ball when a receiver is completing a reception (as mentioned above, there are certain play situations where if there is even slight movement with any part of the ball—including the forearm area, a pass will be considered incomplete even if the receiver actually caught the ball). No art exists that could offer a player the ability to achieve an enhanced grip capability throughout the general arm area, where the ball touches the player (such as the forearm, elbow and bicep area). No advances have taken place to increase one's grip around the wrist area (an area that almost always touches the ball when cradling the ball), nor have there been advances to increase ball control around the elbow or bicep areas.

Providing such a device would certainly allow a player to have a much stronger grip throughout and around the ball, to create a more stable overall handle on the ball, and therefore to significantly advance arm task performance and play execution. Not only would new art offer benefits to running backs, receivers, tight ends and quarterbacks, but they could also increase the performances of kick returners and punt returners, who have to run with the football.

Because no such art exists, it is no surprise that there were a total of 731 fumbles, and the highest quarterback completion rating was less than 70 percent, in the 2010 NFL Season (Official Stat Book of the NFL, 2011). Given the fact that fumbles and incomplete passes persist at the professional level and therefore certainly at the collegiate and amateur levels, one can see that past attempts to solve these problems have had limited success.

An example of another sport in need of control enhancers for the arm area is Volleyball. Here too, ball control is crucial to play performance. Play execution often depends on one's ability to control a ball with multiple parts of your arm, but especially your forearm. Although there are forearm, elbow and even bicep devices that could be used for protection, or simply for aesthetic purposes, no prior art exists that would enhance ball control or grip in said areas.

When an opponent strikes a volleyball to your teams side of the net, the first player to normally touch a ball—when the ball isn't blocked at the net—generally controls the ball with her or his forearm(s). With that first touch contact one must be able to stabilize the ball, and then usually pass the ball to a teammate. Unfortunately, this current method of passing a volleyball can provide inconsistent results. First, the initial contact control is generally with the forearm skin of the player; and without any grip-enhancing device, it can become very difficult to redirect a volleyball with consistent precision. Although the initial contact happens very fast, a control-enhancing mechanism for the arm area would have a significant impact on the sport. Additionally, a more general reason for inconsistencies is in one's inability to grip with one's forearm. Without providing the forearm with some way to better grip a volleyball, inconsistencies in the sport of volleyball will continue to be a long-standing problem.

Not only are there significant needs for grip-enhancing devices for the arm area, in particular the wrist, forearm, elbow and bicep areas, but there is also a significant need for better grip-enhancing devices for the hands as well. Although offering some advantages, using prior art creates disadvantages to often force an individual to choose not to use any grip enhancing device at all.

There are several non-sport activities in need of targeted hand task enhancers for general use.

One growing non-sport hand task in need of enhancers has to do with handling mobile devices, such as, but limited to, tablets, and mobile phones.

For example, texting has become part of everyday life. One often uses one's cell phone to text several times a day. The current method is to simply use primarily the Forefinger and Middle finger to hold the phone, and use your Thumbs to type text from a very small keyboard. This can be a rather difficult task to accomplish if one's Thumb is significantly larger than the individual keys of the keyboard, often resulting in double keying or significant delay in composing the text. There exists an unrecognized need for a device that could be placed on the thumb which would allow the individual to more effectively from texting.

As another example, there are several indoor activities where there is an unrecognized need for hand task enhancers. Activities requiring prolonged hand movements, such as sweeping, mopping, and general house cleaning often require one to grip and/or otherwise control an object. Many choose to conduct these tasks bare-handed often because they don't need all the protection from injury or from grime usually associated with having to use a glove. Most brooms, mops and other household devices however, do not have a grip enhancing component on their handles, leaving an opportunity to provide an non-industrial strength grip enhancer, such as perhaps a gripping means that can enhance only the part of the hand that requires grip enhancers without encumbering the rest of the hand. This could result in significant increase in overall ease of use and therefore increased cleaning performance.

Many basic home repair activities requiring tools, such as hammers and wrenches, are often completed using bare hands (especially when the repair work is done indoors). Many individuals choose not to spend the money on expensive construction gloves, and instead choose to forgo any extra grip enhancers—or protection—and complete the task bare-handed. There therefore is an opportunity to provide a relatively more cost effective and convenient solution whereby an individual could complete these hand tasks.

The use of gloves has other drawbacks, especially when water is involved. Unless one buys expensive swimmers-type gloves at are waterproof, one is usually forced to go without any grip enhancers when conducting activities involving water. Providing, therefore, a cost-effective waterproof grip enhancer would certainly meet a currently unmet need.

Improved art is also desired to support outdoor yard work. Quite often one chooses to use gloves for outdoor yard work. Although it does protect ones hand from injury and grime, at times one finds that they have to take of the gloves momentarily, especially during the hot summer months because of the perspiration building up in the gloves. This inconvenient and unnecessary problem is created because of lack of ventilation in many yard gloves. When the yard work requires intense activity, then the gloves are worth the hassle, but very often the yard work is not very demanding. For example, when one has to control a shovel or rake, the work is often not very demanding but it is very repetitive. The choice that one faces is to either wear a glove for the added grip, but then have to stop a few times to take the gloves off and dry your hands, or choose not to use a glove at all. One result, at times, is that one choose to wear a glove because of a lack of an alternative that might possess many of the advantages of wearing a glove but without any of the disadvantages.

A related issue happens when ones outdoor devices start to wear out. Many shovels and rakes, for example, have a metal face and a wooden handle. Obviously the wood begins to deteriorate much earlier than the usefulness of the metal face. Continuous use, however, might result in one getting splinters on one's hand. The choice then becomes to either discarding the device and buy a new one (an expensive choice) or being forced to wear gloves. An attractive third option, however, would be to develop a cost effective way to tape the handle.

More generally, there are clear indications that an entirely new market exists for targeted grip enhancers that could be attached (using an adhesive means) only on select areas on the hand, leaving other parts of the hand uncovered.

Another particular area where prior art poses significant disadvantages is in any sport where a player's success depends on her ability to both grip as well as feel a ball or object. These players often have to choose between wearing a glove thereby enhancing grip but losing significant feel, or going without a glove thereby maintaining maximum feel but missing the opportunity to enhance one's grip.

In football for example, gloves can be used to enhance performance. The use of gloves in football is so widespread that nearly every football player uses them, with the notable exception of football quarterbacks. You rarely see a quarterback wear gloves, even if just to keep warm. Most quarterbacks choose to play football without gloves. This is largely because prior art consists of generic full-fingered gloves which are uncomfortable and burdensome on a quarterback's dominant (throwing) hand, particularly on those fingers a quarterback places over the football laces. In addition, the full-fingered gloves prevent a quarterback to have any ‘feel’ of the ball.

Playing the position of quarterback without any grip enhancing mechanism, however, can also be an inferior choice. The website Wikihow.com, provides a good description of the conventional way to hold and throw a football. “Throwing the football is simple. Put your non-throwing side foot in front of you. Have your Pinkie, Ring and Middle fingers around the laces with your Index [Forefinger] finger on the strap. Put the other hand up on the ball. Put the ball up by your ear. Twist your hips toward the front foot. Throw the ball at the receiver.” Whereas, the fingers over the laces have a solid grip on the ball—primarily due to the football laces on the ball—the two fingers off the laces (forefinger and thumb) are virtually unsupported and therefore have a relatively weaker grip, creating a weak overall grip on the football.

This weak overall grip becomes more pronounced when added stress is placed on the Thumb or Forefinger. When a quarterback, intending to pass the football, for example, suddenly has to scramble, or if the quarterback ‘pumps’ the ball (goes through all the motions and speed of throwing the ball but doesn't actually release the ball), the grip strength of the Thumb and Forefinger can determine whether or not a quarterback fumbles the ball. Also, if one performs a simple test and wets his/her dominant hand, and then grabs and pumps a football, the Forefinger and Thumb will often move or slip. On a wet football field, during extreme weather conditions (hot or cold), that weaker or looser grip makes for a much more difficult completed pass, less success at throwing a spiral, and inconsistency and inaccuracy in passing.

Under the ‘tips’ section of Wikihow.com, it further describes proper football throwing form: “A proper throw will feel like it's only utilizing the Thumb, Index [Forefinger], and Middle finger. Good release will ‘roll’ off of your Index and Middle finger, to impart more spin; you may snap your wrist through as you follow through to the hip. The other three fingers on your hand stabilize the ball as its being flung. They should not be used to impart spin on the ball. The most important finger to throwing a spiral is the Index finger; it is the finger that holds the most leverage in putting spin on the ball.”

This need to ‘feel’ a ball with a hand has therefore resulted in quarterbacks have to choose between feel or no feel. Although clearly these players would benefit from added grip enhancers on the throwing (dominant) hand, prior art (in the form of gloves) force a quarterback to choose between all feel or no feel. Virtually all quarterbacks have chosen to maintain feel and sacrifice the ability to better grip the football. It is no surprise that quarterback fumbles remain a significant problem in football, even at the highest performance levels, and currently remains an insoluble problem in the sport for amateurs and professionals alike.

Individuals who play basketball also have to ‘feel’ and grip a ball to perform properly, and although they too could significantly enhance performance in controlling a ball, prior art forces them to choose all feel as well, and go without any type of grip-enhancers at all. This insoluble problem therefore also exists in playing the sport of basketball, and these players would substantially benefit from developing a way to maintain feel while increasing grip capabilities in select areas of the hand. More specifically, new art is needed that could offer control enhancers in certain locations of the hand while leaving others areas of the hand uncovered and therefore being able to maintain necessary feel.

In the field of Golf, to be sure, there exists much prior art in the form of gloves for a golfer's weak (non-dominant) hand. In fact most active golf players wear a glove on their weak hand, and go without a glove for their strong hand (if one were to go to any major store to buy golf gloves, they would be sold and packaged in singles—one glove—not sold in pairs). Gloves are prevalent in golf largely because of the role that hand grip and control play in a golfer's overall performance.

Although there exist many types of full-fingered gloves for a golfer's weak-hand, they all attempt to maximize a golfer's weak-hand grip without regard to a golfer's weak-hand feel, and hand coordination needs. It is no surprise, therefore, that prior art consists of full-fingered (all fingers are covered), closed palm (entire palm is essentially all covered) gloves. As a result, a typical golfer must rely on his/her weak-hand to provide most of the grip support, and on his strong-hand to provide all of the ‘feel’ in her golf swing. The current solution to this insoluble problem has been for virtually all golfers to use one and only one glove. This glove is always placed on the weak hand, leaving the strong hand without a glove. There is, therefore, an opportunity to invent a device or method that could offer some ‘feel’ ability for the weak-hand, without significantly diminishing that enhanced grip ability that hand covers offer. This would increase overall hand control of a golfer's club swing, and therefore result in greater success in competition.

Whereas weak-hand support products seem to be crowded in the sport of Golf, there is a long existing need for a device that could offer added support for a golfer's strong-hand without significantly diminishing its ability to adequately feel the golf club. Inventing a solution to this problem could, among other things, allow for greater golf swing control and consistency, and create an entirely new market because golfers currently do not use grip-enhancers on their dominant hand, thereby changing the way that golf is played.

In Golf magazine's April 2005 article titled “Fix your grip. The wrong grip can cripple your swing—Here's the cure”, golf instructor Charlie King provides an overview of how to grip a golf club. “Good golf starts with your grip. The proper hold on the club helps you do three crucial things: Hinge your wrists, control the clubface at impact and support the club throughout the swing. Here are three simple grip tips.” As King continues, his third tip is “both hands; solid at the top. An effective grip sets the face square at the top, with the shaft parallel to the target line. You should feel most of the club's weight in your left Thumb and right Forefinger. Now you're ready to turn it loose.” Although prior art seems to be crowded in offering a hand cover for the weak-hand, to support and better control the club weight placed on the Thumb of the weak-hand, there remains an unmet need for added support on or around the Forefinger of the strong-hand. Additionally, constant swinging of a golf club at real swing speeds often results in soreness on and between the Thumb and Forefinger of a golfer's strong hand (wearing no glove). This soreness can often also come from the rubbing or slipping, between the club handle and the strong-hand, suggesting a need to find a way to increase the grip—as well as protection—of a golfer's strong hand. This is especially important in the sport of golf because even the smallest of slipping—during the golf swing or upon impact of the golf ball—can create enormous inconsistencies and inaccuracies, critical issues in determining overall performance.

Consequently, there are clear indications that an entirely new market exists for targeted grip-enhancers that could be placed only on select areas of the strong (dominant) hand, leaving other parts of the strong hand free to feel. In particular there remains an unrecognized problem and an unmet need that would provide multiple benefits, such as better overall grip, more coordination with both hands, as well as some protection from any constant grip slipping, during the practice or play of golf, and in various other sports activities.

In the sport of Basketball, there exists no prior art when it comes to grip-enhancers to enhance the performance of over 100 million individuals who play the sport. Although there are several multisport gloves in the market today, virtually no one uses gloves when playing basketball. A primary reason why basketball players choose not to use gloves, as mentioned briefly above, is like the issue with football quarterbacks, basketball players often need to be able to both grip and feel the ball. Although many hand tasks require a good grip, no art currently exists that would provide these players with enhanced grip capabilities, or enhanced protection, without having to sacrifice the critical ability of being able to properly feel the basketball as well.

One clear hand task in basketball is in shooting the basketball with the intention of making a score or basket. Conventional jump-shot shooting form requires, among other things, that the player hold the basketball largely with the fingertips of both hands, and creating a small opening—or a shooter's gap—between the ball and the palm area of the player's strong-hand. No grip enhancers exist that would increase the gripping abilities of a players fingertips and leaving the rest of the hand free to feel.

No prior art exists that would provide an athlete with the ability to have enhanced control when dribbling a basketball. Proper dribbling form is to rarely, if ever, look at the ball while dribbling said ball—thus one of the critical reasons to being able to maintain high ‘feel’ ability. Without any extra grip-enhancers however, it is difficult to maintain stable control of the basketball.

A typical game—even a professional game—often can have as many as 30 turnovers (combined), so offering art that could increase ball control while dribbling, passing or even catching a basketball could significantly enhance performance by, among other things, minimizing turnovers. Minimizing turnovers, for example, could be achieved by offering better ball control while dribbling a basketball. Those players playing the position of Guard may benefit from added grip support especially because they may need to dribble, at least briefly, with either hand.

Although athletes playing the position of Forward or Center would also benefit by enhanced dribbling abilities, most of the turnovers caused by Forwards and Centers are often the result of dropping passes thrown to them, or from making a bad pass. Offering art that would enhance the ability to better pass or catch a basketball could therefore also enhance overall performance for anyone playing the sport of basketball.

Another hand task in basketball is in slamming a basketball through a basketball hoop (commonly known as slamdunking). When one slamdunks a basketball it is often done with force and almost always involves striking a metal basketball hoop with at least one of your hands (mostly impacting the fingers). For Forwards and Centers especially, this hand task can quickly take a toll on their fingers if their fingers are not somehow protected. The current—and only—method of enhancing this hand task is by applying basic sports tape to select finger joints. This current method and product has significant shortcoming that often result in inferior execution. First, if one tapes parts of ones hands with the prior art, they will lose any grip capabilities in that area, which could then result in mishandling the basketball (a significant issue with Forwards and Centers). Second, the tape doesn't provide for any extra padding along the taped area; the area is simply protected by the thickness of the tape and the number of times one wraps the tape on their fingers. Better devices and methods of providing added protection while slamming while not sacrificing ball control would significantly impact the sport of basketball.

Other general hand task challenges that are in need of a better solution have to do with basketball players who injure, in some way, their hand. In this situation the player has to tape her hand, especially when injuring a finger. The result again is a decrease in ball control and limited protection. To protect the injury, most players will choose to tape the finger and try to adjust. Loss of dribbling control often results, as does loss of ball control when preparing to shoot the ball (as seen when the ball slips out of the players hand and flies up in to the air as they are shooting the ball).

### DETAIL DESCRIPTIONS OF THE INVENTION

In general the present invention offers task enhancers for the arm, the hand and in select areas of the arm and/or the hand. These arm and hand task enhancers can be attached to the individual, and thereby increase, among other things, the grip capabilities of that area. The present invention offers a general purpose gripping means that could be attached to an individual using an adhesive means on one side the present invention, and comprising a gripping means, on the other side of the present invention.

Embodiments of these control enhancers can, for example, increase the overall performance in arm and/or hand task activities, such as but not limited to, by allowing an individual to better control a ball, object or child. Embodiments can offer grip enhancing capabilities for the arm area, in particular, for example, the wrist, forearm, elbow and bicep areas, as well as for the hand, including the fingers of the hand, and methods thereof. Additionally, embodiments could also find offer unique benefits by offering the ability to also be attached to objects and devices as temporary, targeted grip enhancers.

The adhesive means of the present invention could comprise various types and strengths of adhesives to ‘stick’ or bond to an individual. Adhesives used, for example, by ASSURED LONG LASTING ADHESIVES, or BAND-AID BANDAGES, could provide sufficient capability for many embodiments of similar size as the above mentioned popular products. For larger embodiments, such as the forearm patch embodiments, adhesives used by SALON PAS patches could provide the necessary bonding capabilities.

The gripping means, as claimed in claim 1, can comprise of various grip-enhancing materials, forms, and designs including, but not limited to, foams, fabrics, PVC dots, perimeter patching designs, linear and non-linear grooves, or combinations thereof.

The present invention may comprise of various colored and multi-colored materials, forms and designs including, but not limited to, fluorescent coating. Such as, for example, trademark designs or images for aesthetic purposes. Embodiments may comprise various types of materials, forms, and designs including, but not limited to, stretch materials and designs, mesh fabrics, recycled and flexible materials, cottons, polyester, rayon, spandex, fleece, leathers and synthetic leathers, rubbers, plastics, or combinations thereof. Embodiments may comprise various weather-resistant and perspirant-resistant materials, forms and designs including, but not limited to, water-resistant materials or hole designs for moisture management, or combinations thereof.

Arm task embodiments of the present invention can assist an individual more effectively perform activities outside of sports activities.

One non-sport arm task activity where forearm embodiments of the present invention would meet an unmet need would be in the food services industry. For example, a waiter (or other food service provider) often has to use a bus tray, placing the bus tray on his forearm and gripping it on one edge with his hand. The forearm embodiment could be placed on the waiters forearm. This embodiment could comprise a series of non-linear grooves to maximize grip and control. Furthermore, this embodiment could also provide added comfort as well as protection, by comprising of thick padding along the area most likely to touch the bus tray. This embodiment would have an adhesive throughout one side of the embodiment for maximum hold and attachment to the forearm. The other side of the embodiment, as mentioned above, would have the grip-enhancer. This particular embodiment would offer a middle layer, comprised of a padding element (see FIG. 2).

Another non-sport activity in need of arm task enhancers has to do with holding or cradling a child. When someone cradles a child in their arms, the individual—usually an adult—generally places the head of the child on the adult's forearm, elbow and/or bicep area. An embodiment of the present invention could comprise an arm patch, covering most of the entire arm area. This oval or rectangle embodiment would provide, among other things, padding throughout so as to add comfort for the child and some protection for the adult. It could be made from cotton and an elastic fiber. The outer surface could comprise of a soft, silk or silk-like material. The thinkness of the padding could vary as well. One could develop a variety of middle cushion layers, ranging from thicker or thinner paddings to different colors.

One sport where said present invention will clearly enhance performance is in the sport of football. For example, one particular unmet need that a grip-enhancing device for the arm area will satisfy, will be with receivers. One embodiment of the present invention comprises fingertip patches will allow a receiver to increase his overall ball grip and therefore control of a football especially when catching a football and attempting to complete a reception. These embodiments, one for each of the players ten fingers, could be designed to minimize movement on impact. The embodiment could also have PVC dots, perhaps forming an oval shape, along the outer surface. The football player would place a fingertip embodiment on to each of his fingertips (See FIG. 1). This embodiment could be made of natural rubber or natural rubber latex. For a receiver, this and other embodiments would also allow the player to increase receptions and decrease fumbles, by being able to better cope with added stress factors, factors which would otherwise make for an unstable overall grip.

Embodiments in combination could offer greater consistency and enhanced performance would comprise two embodiments, one covering the elbow area and the other attached to the bicep, with a grip-enhancing design such as several grooves throughout these embodiments. These embodiment would enhance a player's ability to better absorb impact from the ground or from a defender—without losing control of the ball. Additionally, this embodiment would minimize the possibility of the ball moving, once in the receivers grasp because, among other things, grip capability has been enhanced throughout the arm. These embodiments would, of course, be used to better grip said football throughout the arm area. These embodiments could be crafted with a blend of polyester and LYCRA spandex for a tight fit. The width of these embodiments could for example, generally begin around one to two inches but could just as easily range beginning from 3 inches and more.

Embodiments could also significantly improve the performance of running backs, or any player who runs with a ball. One embodiment of the present invention, comprising wrist patches would allow a player to significantly increase his ability to control or cradle a ball when running. This embodiment could be placed on the wrists to help stabilize control, thereby supplementing a grip of any glove that might be currently available and in use. This embodiment could be made of material used to manufacture standard sports tape. The embodiment could offer PVC dots to provide enhanced grip capabilities, and adhesive used in sports tape, to provide the sticking means to attach to the wrists.

Embodiments could also be used as a grip enhancer around the elbow area. Specifically, two could be used; one could be placed just below the elbow area and the other just above the elbow area, to almost literally grab one end of the football with the elbow. These embodiments could be comprised of rubber, much like that used to manufacture a standard sports glove. Clearly, this would create a much greater, more stable overall grip because one would now have grip enhancers that one could use to increase ones grip on both ends of the football, with a glove on the hand holding one end of the football, and the grip enhancers on the elbow area holding the other end of the football.

Other sports, such as Rugby, could benefit from many of the described embodiments for many of the same reasons.

Embodiments of the present invention can offer significant and substantial benefits by offering the ability to better control a ball in select areas of the arm. For example, embodiments could be placed on primarily only the forearm area, allowing the player to have significantly enhanced control over a ball, much like a glove can help a players' hand better control a ball. In addition, these forearm embodiments offer new and surprising results when used in the sport of volleyball: they allow a player to impart much more spin on the volleyball—an added feature which could revolutionize the sport This ability to impart significant spin on the ball could make it easier to control the ball upon contact, to strike a ball, to get the ball to a teammate, and to redirect the ball.

One particular embodiment could comprise of an attachable forearm embodiment. One could consider it an attached glove for the forearm, being made of materials similar to that of a typical hand glove, and offering similar benefits to that of a hand glove as well. Specifically, it could be made of a stretch, moisture-resistant fiber, with grooves along most of one side, and elastic material on the other side. As a result, one would now have significantly more ball control during volleyball practice or game play. After the activity was completed, one could then simply take off this embodiment, and perhaps store it for future play.

Hand task grip enhancers could certainly assist an individual more effectively perform non-sport as well as sports activities, or indeed for general use.

One growing hand task in need of enhancers has to do with handling mobile devices, such as, but limited to, tablets, and mobile phones. For example, texting has become part of everyday life. One often uses her cell phone to text several times a day. The current method is to simply use primarily forefinger and middle finger to hold the phone, and use your thumbs to type text from a very small keyboard. This can be a rather difficult task to accomplish if one's thumb is significantly larger than the individual keys of the keyboard, often resulting in double keying or significant delay in composing the text. The exists an unrecognized need for a device that could be placed on the thumb which would allow the individual to more effectively from texting.

Many individuals are now using the very popular mouse-device-free tablets. These devices allow you to touch the screen and use your fingers, as the mouse. More specifically, one uses the tablets by placing one's thumb and, at times, the forefinger. One would use these fingers, and others at times, to scroll, text, and otherwise manipulate the screen. One result is that you often have finger prints all over the tablet screen. Providing a device which could be placed on select fingers could not only eliminate these fingers smudges but could also assist in better overall control—and grip—as well as more effectively controlling your desired screen destinations, while leaving other areas of your hand uncovered and unencumbered. There is also an unmet need for providing a device that would allow one to more effectively grip and/or more comfortably control said devices.

Other areas where embodiments would more effectively increase performance include, but are not limited to:
Indoor activities where a glove is often burdensome,
Gripping daily household items like twisting off a tight lid,
Placing on just thumbs and forefingers when doing a manicure,
Gripping a hammer or mobile device (such as tablets, cell phones).
General construction work

As one can see, embodiments of the present invention has all the advantages of the prior art and none of the disadvantages. Moreover, embodiments are also better than glove because offers less waste of materials.

In addition, one can use embodiments to wrap an object or device, in order to provide general enhanced grip. One can attach a grip enhancer on, for example:
Walkers—on the walker or on the hand.
Part of a golf club, instead of having to regrip the entire handle
An old shovel or rake whose wooden handle is beginning to splinter

Embodiments would not be seen as permanent grip enhancers, which is to say that they would not be expected to last for more than a year. Embodiments could, however, be designed to last more than a few months, days, or hours.

Individuals engaged in a sports activities involving the hand, could also clearly benefit from embodiments of the present invention. One sport where hand task enhance embodiments of the present invention will clearly enhance performance is in the sport of football. For example, one particular unmet need that finger joint embodiments will satisfy will be with football quarterbacks (though receivers may enjoy this, too). In the book “coaching football successfully,” by Allan Trinkle (2001) “quarterback mechanics and ball-handling skills are vital for offensive success and consistency.” One embodiment of the present invention comprises circular patches for the joints of the dominant hand. This embodiment would allow a quarterback to increase his ball grip and overall control of a football, while still allowing some vital finger feel of the football as well. This embodiment could be made of a natural rubber, having PVC dots throughout the finger joint embodiment. Because football is often played outside, embodiments might be made of moisture-resistant fibers as well. The benefits to the user df this embodiment would include: better overall grip and better control in holding and throwing a football, higher throwing accuracy, and less fumbles. This embodiment would take into account the benefits of the laces on a football, and give a quarterback the unique ability to grasp a football over the football laces on a football, with the comfort and feel of not having a glove, while adding the support that a glove might provide, over the thumb and forefinger. No prior art offers this unique type of support. This embodiment would therefore increase grip capability on select fingers, while leaving the rest of the hand uncovered and able to maintain the necessary ‘feel’ of the ball, something that prior art cannot do. The resulting stronger overall grip should make for a much more higher completed pass ability, more success at throwing a spiral, and generally consistency and performance in ball handling and control.

In addition to offering greater throwing accuracy and consistency, these and other embodiments could also help minimize quarterback fumbles by adding gripping support when completing other quarterback tasks, such as when ‘pumping’ the football, scrambling from being tackled, and even when catching and passing the football, especially when in ‘shot gun’ formation.

Another sport where the present invention will meet an unmet need is in the sport of golf. Many embodiments can be used in conjunction with each other to uniquely solve problems that have up until now been solved. As mentioned, gloves are not used on a golfer's dominant hand because of the structural limitations of this prior art. For example, a golfer may want to enhance the grip around some fingers but might want the fingers uncovered as so to maintain critical feel sensitivities. New art in the form of multiple finger task enhancers to support a golfer's strong hand would now allow a golfer to select which fingers she wants covered and which she wants to remain uncovered. The benefits of this significant and substantial physical difference in this new art could change the way that golf is played by among other things, offer the golfer a more stable overall grip, better control and enhance performance. For example, one embodiment for a golfer's strong hand comprises finger joint embodiments, ones that each cover a joint of the dominant hands Thumb, Forefinger and Middle finger, leaving the Ring and Pinkie fingers uncovered. The ‘feel’ and coordination tasks could therefore be maintained with the Ring and Pinkie fingers by leaving these fingers uncovered, while enhancing the overall grip of the strong hand by covering the strong hands Thumb, Forefinger and Middle fingers. This embodiment therefore offers the golfer offers a much improved method of playing the sport of golf by using grip enhancers not just on the weak hand but also on the strong hand as well, without limiting the feel and coordination requirements in a proper golf swing.

Currently, only full-fingered gloves exist for golfers, regardless of one's preferred golf grip. One very popular grip, for example, is call the ‘interlocking grip.’ When one uses this grip, the Forefinger of the golfer's weak hand is placed over her strong (dominant) hand. With this grip, clearly the role of the weak hands Forefinger has less to do with grip and more with coordination and feel on the strong hand, to more effectively control the golf swing and provide greater golf swing consistency. There is, therefore, no need to cover the weak hands Forefinger, and covering the Forefinger (with a glove, for example) can actually diminish the forefinger's ability to properly feel the other hand, thereby diminishing the ability to coordinate a consistent, proper golf swing. Embodiments of the present invention. could offer significant improvements to gloves by allowing a golfer to self select which fingers she wants covered and which ones she doesn't. For example, she could choose to have finger embodiments to cover all the fingers on her weak hand, except the Forefinger. These embodiments could be made of the same material used to make a typical golf glove, such as rubber or synthetic rubber. Alternatively, the golfer may simply prefer to simply add a finger embodiment on just the Pinkie finger of her dominant hand. This idea clearly also would apply to someone gipping a golf club with the ‘overlapping’ and ‘full-fingered’ methods.

Another possible outcome in the way golf is played may be in how one handles a golf putter. Current proper form is not to use a glove. If one were to watch a professional golf tournament, for example, nearly all golfers would use one glove (placed on their weak hand) that they would use to grip a club for the tee shot swing, the fairway swing and even the short, approach swings. Once the golfer was ready to use their putter and putt the golf ball, they always take their glove off, deciding to grip the putter with both hands uncovered (without any sort of grip enhancer, such as a glove or patch). These finger embodiments could be made of a softer mesh fabric and perhaps a smoother surface but also with a griping coating or compound, such as PVC coating, and would now allow a golfer to choose, if she prefers, to increase the grip she has on her club, but only modestly. For example, the golfer could choose to keep her overall feel of the putter very high by not using a glove on either hand. However, she could then decide to use a Thumb embodiment (for her dominant hand) and a Pinkie finger embodiment (for her weak hand), thereby also modestly increasing her grip at both ends of her hand grip. Even a slight increase in control of a putter can make a very significant and substantial difference in performance, especially if one ‘drives for show and putts for dough.’

Another sport where hand task grip enhancer embodiments of the present invention would meet unmet needs would be in the sport of basketball. Individuals who play basketball have to ‘feel’ as well as control a ball to perform effectively (as mentioned previously), and although they too could significantly enhance performance in controlling a ball by using grip enhancer, prior art forces them to choose between all feel (and therefore, no glove) or no feel, and go without any type of grip enhancers. These players would substantially benefit from developing a way to maintain feel while increasing grip capabilities in select areas of the hand, thereby solving this significant issue. This insoluble problem can now be solved by embodiments of the present inventions.

One embodiment that would help a player shoot better comprises finger tip circular patches, while the rest of the hand, including the palm area, can remained uncovered. These embodiments could comprise of an adhesive on one side (all embodiments have an adhesive on one side) and non-linear grooves on the other side of the embodiment. These embodiments could be made of the moisture-resistant fabric. For all basketball players, but especially for those who have trouble shooting free throw shots or shooting outside of the perimeter, the uncovered palm area helps to remind them that the basketball should rarely touch the palm area when properly shooting a basketball (if the basketball touches the palm area, then there is no shooter's gap and is therefore considered improper shooting form. Because proper dribbling form also discourages the ball touching the palm area, covering the palm area with, say a glove, would not only be relatively useless in basketball, but also a waste of resources. My embodiments would allow a player to avoid covering her palm area and instead concentrate her enhanced gripping abilities in the most important area when it comes to shooting; the fingertips. The result would of course be a better overall grip and a significantly superior alternative to the current art of basic multisport gloves that cover the hand completely forcing the basketball player to lose much of the critical requirements of being able to feel the ball as well.

Embodiments could also satisfy the necessary requirements in dribbling a basketball as well. According to the book “Basketball for High School Players and Coaches,” (1955) Carl Bachman describes proper fundamentals of basketball dribbling: “Certain fundamentals apply to all phases of ball handling: Looseness of finger and wrist action is important, practice spinning the ball on fingertips; a basketball should never touch the heel of the hand and seldom, if ever, touch the palm.” Fingertip embodiments of the present invention could also help a dribbler develop and use proper dribbling form, especially on her weak-hand. This embodiment would more generally help any player, and likely could be used, for example, by those playing the position of Guard while practicing proper dribbling on their strong-hand, as well as Forwards and Centers during actual game play for added support on their weak-hand.

Another embodiment for enhanced general basketball play is in the form of oval finger joint embodiments. All five fingers are all individually, and separately covered. Additionally, the palm area would therefore be uncovered. This embodiment would give a basketball player the ability to better catch a ball, thus eliminating the consistent problem often found in Forwards and Centers losing control of passes. The embodiment would also provide a player with a stronger grip on the ball when passing a ball as well as provide some moisture management control, thus minimizing turnovers often caused by passers, especially in Guards.

Although many hand tasks require a good grip, no art currently exists that would provide these players with the ability to maintain some grip capabilities while at the same time trying to protect the hand from injury, or trying to protect an already injured hand. Finger embodiments of the present invention would allow a player to be able to apply some sort of protection on select fingers, without having to sacrifice the critical ability of being able to properly feel the basketball.

For example, one common hand task in basketball is slamming a basketball through a basketball hoop (commonly known as slamdunking). When one slamdunks a basketball it is often done with force and almost always involves striking the metal basketball hoop with at least one of your hands (mostly impacting the fingers). For Forwards and Centers especially, this hand task can quickly take a toll on their fingers if their fingers are not somehow protected. The current—and only—method of enhancing this hand task is to apply tape to select finger joints. This current method and product has significant shortcoming that often result in inferior execution. First, if one tapes parts of one's hands with tape that has been invented, the player will lose any grip capabilities in that area, which could easily result in mishandling a basketball. Second, the tape doesn't provide for any extra padding along the taped area; the area is simply protected by the thickness of the tape and the number of times one wraps the tape on their fingers. One finger task enhancing embodiment, therefore, could comprise of finger joint embodiments for the base joints of the hand. These base joint embodiments would comprise of a middle padding layer made of, for example foam about ¼ inch in thickness. Again, one side would offer an adhesive element and the other side would offer a gripping element (such as PVC dots). Among the results would be a better device and method of providing added protection while slamming, and not sacrificing ball control.

A secondary but still important feature is that this embodiment will help players slamdunk a basketball by helping the player to ‘palm’ the ball. This feature would provide added grip support and greater performance in slamdunking.

Other general hand task challenges that embodiments would help solve has to do with basketball players who injure, in some way, their hand. This situation is very common in basketball, where a player has to tape her hand after, say, injuring a finger. The result again is a decrease in ball control and limited protection. These hand task enhancer embodiments could also allow a player to essentially ‘tape’ her finger by sticking one of these embodiments on her hand. Embodiments, as mentioned above, could also include a padding interior, which might result in the player not needing tape to protect an injury. These finger embodiments would be substantially superior because these embodiment would, for example, be able to protect a finger injury (with the padding element) while not diminishing grip, because of the gripping element on the outer surface of said embodiments.

In general, the hand task enhancer embodiments of the present invention can generally be used in conjunction with any type of hand task activity and/or sports play. As discussed, they offer an individual with the opportunity to increase overall hand task performance. Maintaining or increasing overall control, for example, can provide many benefits to a user of these, and other embodiments. Among the many benefits of the hand task enhance embodiments are that they:
Allow an individual to maintain or increase control of a ball or object
Offer the ability to grip as well as feel a ball and/or device;
Provide the unique solution for players who desire better grip capabilities only in select areas
Allow a player to protect an injury on a finger without losing significant grip capabilities
   disadvantages that using prior art sports tape would create if not used in conjunction with these embodiments
Give a means for players who want added protection to do certain activities, such as slamdunking a basketball, without losing grip capabilities that the current art sports tape would create.
Provide a player with the added convenience to select which fingers to cover and which fingers to remain uncovered, simply even just to maximize overall comfort during sports play
Offer a more stabilizing overall grip of a ball or object, by conveying grip-enhancers to select locations of the hand.

Additionally, these embodiments can provide:
Improved performance in hand task execution
improve overall grip
improve stability of overall grip throughout the hands
more control
more consistency
more hand coordination by adjusting enhancers to match one particular golf swing
less ball mishandles

These are among the many benefits of the present invention, and is not to be construed as limitation of the benefits nor their legal equivalents

Although the description of the present invention only discussed a few embodiments, it is understood that other non-sport and sport activities might benefit as well from the present invention and its legal equivalents. In addition, only some embodiments have been discussed and in no way is intended to limit all the various embodiments and other embodiments that the present invention provides, such as but not limited to, different designs. These embodiments can be used by men and women, boys and girls, playing any position in sports, as well as those whose dominant hand is the right hand or the left.

There has thus been outlined, rather broadly, the more important features of the invention in order that the detailed description thereof that follows may be better understood, and in order that the present contribution to the art may be better appreciated. There are, of course, additional features of the invention.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and in being used in other ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTIONS OF THE DRAWING

It is expressly understood that the following descriptions and drawing are for illustration purposes only, and in no way are intended to limit the scope of the present invention and its various embodiments. For example, the drawings are of drawings of embodiments for the left hand but can easily be created for the right hand, and can be used by men and women, boy and girls.

FIG. 1 is a drawing of several hand task embodiments, circular patches.

FIG. 2 is a drawing of an embodiment for the forearm.

### DETAILED DESCRIPTION OF THE DRAWINGS

It is expressly understood that the drawings are for the purpose of illustration and description only and are not intended as a definition of the limits of the invention:

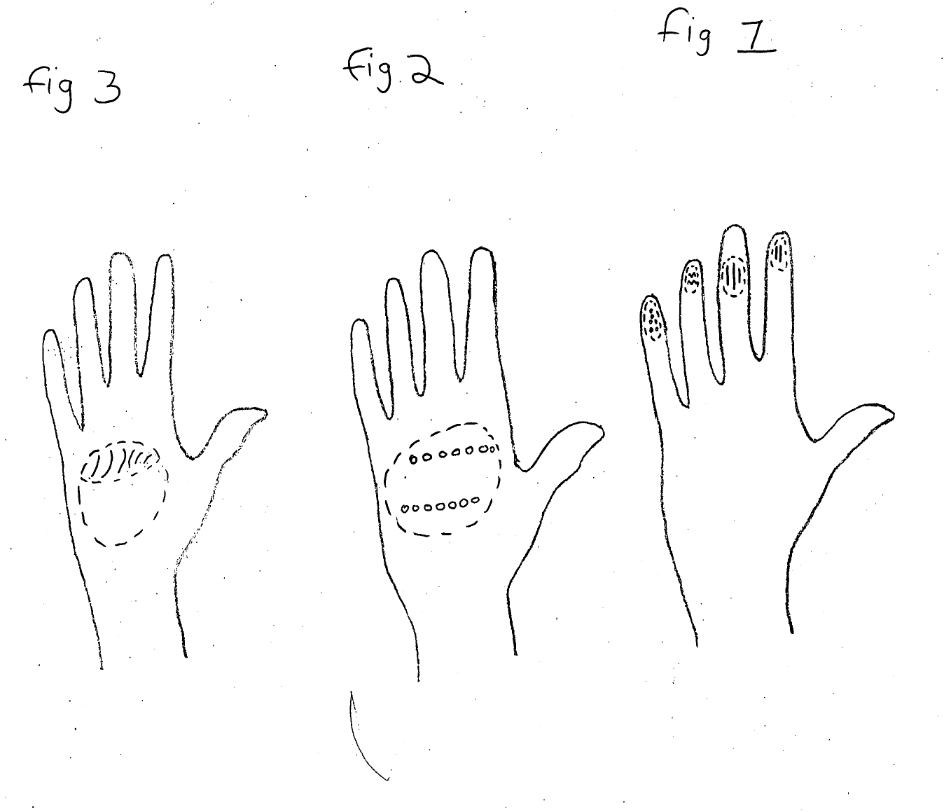

In FIG. 1, the present invention is shown as various grip-enhancing embodiments for the finger. These particular embodiments are made of natural or synthetic latex. The pinkie finger embodiment comprises an adhesive element on one side 10 attached to the pinkie finger. It has a gripping element 11 of horizontal grooves. The ringer finger has an adhesive element used in standard sports tape 12 and has non-linear gripping grooves. The middle finger also has standard adhesive used with, say, BAND-AID BANDAGES, and offers vertical grooves 13. The forefinger offers an adhesive used in ASSURED LONG LASTING ADHESIVE BANDAGES, and comprises of vertical gripping grooves.

In FIG. 2, this particular embodiment can be used to enhance the arm tasks associated with holding or cradling a child. It can be made of polyester and cotton 20 for superior comfort. It also has padding throughout the middle layer 21, to allow the child to be held in comfort. This added padding could also provide added protection for the adult who is cradling the child.

Embodiments could also be made of silk or silk-like materials to further increase a child's cradling experience. This embodiment could also act as a wrist embodiment. The wrist embodiment could be made of a natural rubber latex, with nonlinear grooves 22 to increase the grip in the wrist area. For example, when a football player begins to run with a football, the football is griped usually by one hand, is placed on the wrist and forearm of the arm, and then he begins to run, while swinging his hand—and the ball—while running. This embodiment would help control the ball around the wrist area.

I claim:

1. A general purpose predetermined shape means that could be attached to an individual or object, comprising:
a. A gripping element on one side of said predetermined shape means
b. An adhesive element on the other side of said predetermined shape means
c. Not intended to be a permanent attachment

2. The means, as claimed in claim 1, wherein said gripping element comprises designs such as grooves and criss-cross, PVC dots, gripping materials, or combinations thereof.

3. The means, as claimed in claim 1, further comprising moisture-resistant and weather-resistant materials, coatings rubbers, latex, ntrile, and elastics.

4. The means, as claimed in claim 1, further comprising a middle layer, comprised of paddings or other forms and foams and materials intended for protection and/or comfort.

5. The means, as claimed in claim 1, wherein said adhesive side of said predetermined shape means further comprises a medicated element.

6. The means, as claimed in claim 1, wherein said predetermined shapes, comprises various circular, oval and non-oval patches.

7. A method for slamdunking a basketball comprising the following steps:
a. Providing a general purpose predetermined shape means that could be attached to an individual or object, comprising, a gripping element on one side of said predetermined shape means, an adhesive element on the other side of said predetermined shape means, not intended to be a permanent attachment
b. Attaching said general purpose means onto joints of the basketball players hand or hands,
c. Grasping a basketball with said hand or hands,
d. Slamdunking said basketball through a basketball hoop

8. A method of gripping a golf club comprising the following steps:
a. Providing a general purpose predetermined shape means that could be attached to an individual or object, comprising, a gripping element on one side of said predetermined shape means, an adhesive element on the other side of said predetermined shape means, not intended to be a permanent attachment
b. Attaching said general purpose means on to the finger joints of a golfer's hand or hands,
c. Gripping a golf club using any preferred golf grip,
d. Swinging said golf club

9. The use of the means, in accordance with the claim in claim 1, to dribble a basketball

10. The use of the means, in accordance with the claim in claim 1, to shoot a basketball

11. The use of the means, in accordance with the claim in claim 1, to play the sport of golf

12. The use of the means, in accordance with the claim in claim 1, to grip a tennis racket

13. The use of the means, in accordance with the claim in claim 1, to cradle a baby, child or infant

14. The use of the means, in accordance with the claim in claim 1, to cover a wound

15. The use of the means, in accordance with the claim in claim 1, to grip a baseball

16. The use of the means, in accordance with the claim in claim 1, to run with a football
